(19) United States
(12) Patent Application Publication
(10) Pub. No.: US 2013/0258805 A1
(43) Pub. Date: Oct. 3, 2013

(54) METHODS AND SYSTEMS FOR PRODUCING COMPOUNDED ULTRASOUND IMAGES

(71) Applicants: Jens Munk Hansen, Copenhagen N (DK); Svetoslav Ivanov Nikolov, Farum (DK); Jacob Kortbek, Odense NV (DK); Jørgen Arendt Jensen, Horsholm (DE)

(72) Inventors: Jens Munk Hansen, Copenhagen N (DK); Svetoslav Ivanov Nikolov, Farum (DK); Jacob Kortbek, Odense NV (DK); Jørgen Arendt Jensen, Horsholm (DE)

(73) Assignee: B-K Medical ApS, Herlev (DK)

(21) Appl. No.: 13/879,013

(22) PCT Filed: Oct. 10, 2011
(86) PCT No.: PCT/EP2011/067648

§ 371 Date: Jun. 10, 2013

Related U.S. Application Data
(60) Provisional application No. 61/393,196, filed on Oct. 14, 2010.

(30) Foreign Application Priority Data
Oct. 11, 2010 (PA) 201000924

(51) Int. Cl. A61B 8/14; G01S 15/89

(52) U.S. Cl. CPC A61B 8/14; G01S 15/89
USPC 367/8

(57) ABSTRACT

Disclosed is a method for producing compounded ultrasound images by beamforming a first and a second low-resolution image using data from a first ultrasound emission, beamforming a third and a fourth low-resolution image using data from a second ultrasound emission, summing said first and said third low-resolution image creating a first high-resolution image and said second and said fourth low-resolution image creating a second high-resolution image, wherein the method further comprises computing a first envelope image for said first high-resolution image and a second envelope image for said second high-resolution image, and processing said first envelope image and said second envelope image creating in a first compounded high-resolution image.

### FIELD

This invention generally relates to ultrasound imaging, more particular to methods for producing compounded ultrasound images.

### BACKGROUND

A major benefit of clinical ultrasound is the ability to capture the dynamic anatomy in real time. The diagnostic capability of ultrasound imaging is dependent on the properties of the imaging system in terms of resolution, contrast, suppression of speckle noise, and frame rate. Improving diagnostic capabilities is challenging since these properties are in essence of conflicting nature.

Compounding may be used for speckle suppression. In clinical ultrasound imaging the displayed images have an image overlay with a grainy appearance called speckle. Speckle does not represent physical structure but is a result of diffuse backscattering from tiny tissue structures much smaller than the wavelength of the ultrasound pulse. Since speckle is not assigned a clinical diagnostic value and only perceived as an unwanted imaging artifact speckle is often denoted as speckle-noise. The appearance of speckle makes it difficult to resolve small differences in echogenecity and thus reduces the contrast and limits the ability to display cysts. Different ways of eliminating speckle has been proposed, and one method is compounding. Speckle patterns are related to the viewing angle, at which a given region of tissue is scanned, and the most common method for speckle reduction is to use angular compounding. An image is created by transmitting and receiving beams perpendicular to the transducer surface. Each beam is created by exciting a sub-set of transducer elements to create a transmit beam. The echoes are received with the same or another subset of transducer elements, and an image line is formed along the propagation direction of the transmit beam. After a line has been beamformed, adjacent transmit elements are excited to create a new transmit beam, and a new receive beam is formed in the same direction. The process is repeated till an image is scanned. Then a new image is created in the same sequential manner. The beams are this time steered at an angle. A number of such images are scanned sequentially. The beams from the images are envelope detected and scan-converted. The envelope detected and scan converted images are combined together to suppress speckle. The process is known as spatial compounding or angular compounding.

U.S. Pat. No. 4,604,697 discloses synthetic transmit aperture imaging. The principle of synthetic transmit aperture imaging is to acquire backscattered data with different phase and amplitude information which will allow to reconstruct the image using, for example, delay-and-sum beamforming. Spherical waves are used to scan the region. First a spherical wave is created such as its origin is at the one end of the transducer. The wave propagates in the whole region under investigation and is back scattered to the transducer. Then the acquisition is repeated, but this time the origin of the spherical wave is positioned at another spatial location. The process is repeated until the whole transmit aperture is spanned by the centers of origin of the spherical waves. At every emission a full image is created by delay and sum beamforming. These images have a low resolution since beamforming is performed only in receive. By combining the individual images a resulting image having a high-resolution is created. This proposed method improves the resolution but does not solve the problem of speckle suppression.

U.S. Pat. No. 6,508,770 discloses a method for speckle suppression which is called aperture compounding. In this method a beam is transmitted and received along a scan line. The acquisition is done sequentially with first and second apertures, where the center of first aperture is different from the center of second aperture. The compounding effect is achieved by adding the envelope-detected data from first and second aperture. The method cannot achieve the full potential of compounding for the whole line, and the frame rate is limited by the sequential nature of the acquisition.

US20090069693 suggest the use of the principles of retrospective dynamic focusing for spatial compounding. In their work a line is illuminated by a small set of transmissions. The data is processed to achieve retrospective dynamic transmit focusing. The compound effect in this approach is limited because of the focused transmissions and the line-based beamforming. Further the frame rate is limited by the sequential nature of line-based acquisition.

It remains thus a problem to provide an improved method and/or system for reducing the unwanted effects of speckle in ultrasound images.

### SUMMARY

According to a first aspect, there is provided a method of producing compounded ultrasound images by beamforming a first and a second low-resolution image using data from a first ultrasound emission, beamforming a third and a fourth low-resolution image using data from a second ultrasound emission, summing said first and said third low-resolution image creating a first high-resolution image and said second and said fourth low-resolution image creating a second high-resolution image,

wherein the method further comprises computing a first envelope image for said first high-resolution image and a second envelope image for said second high-resolution image, and processing said first envelope image and said second envelope image creating in a first compounded high-resolution image.

Consequently, there is provided a method capable of producing high quality ultrasound images with a high frame rate. By beamforming two images for each ultrasound emission and further combining images from different emissions, resulting images with both a high resolution and reduced unwanted speckle effects may be provided.

Each low-resolution image may have any size; e.g. it may comprise a single image line or a plurality of image lines. In some embodiments, each low-resolution image comprises at least 2, 5, 10, 20, 50, 100, or 500 image lines. A low-resolution image may be defined as an ultrasound image created by processing data originating from a single ultrasound transmission. The first and the second low-resolution image may be created by using any beamforming method, e.g. a conventional delay and sum beamformer. A high-resolution image may be defined as an ultrasound image created by processing data from a plurality of emissions.

The ultrasound emissions may be focused emissions where the ultrasound wave front converges towards a focus point positioned in front of the ultrasound transducer, e.g. by transmitting an ultrasound signal from a group of transducer elements where the ultrasound signals transmitted from the transducer elements of the central part of the group are delayed relative to the ultrasound signals transmitted from transducer elements of the outer part of the group, an unfocused ultrasound emission where an approximately plane wavefront is created, e.g. by transmitting from a plurality of transducer elements coherently, or a defocused ultrasound emission where an ultrasound wavefront is created that converges away from a point positioned at or behind the ultrasound transducer, e.g. by transmitting an ultrasound signal from a group of transducer elements where the ultrasound signal transmitted from the transducer elements of the outer part of the group is delayed relative to the ultrasound signal transmitted from transducer elements of the central part of the group.

The transmitted ultrasound beam for the first ultrasound emission may differ from the transmitted ultrasound beam for the second ultrasound emission with respect to its geometrical position and/or shape and/or frequency content. Transmitted ultrasound beams at different geometrical positions and/or different shapes and/or with different frequency content may be generated by selecting different sub groups of transducer elements and/or using different apodization functions and/or using different excitation signals having different frequency contents.

The low-resolution images may be weighted before they are summed into high-resolution images. The weight may be a single weight for all image points in a low-resolution image or a particular weight for each image point in a low-resolution image may be used. The weights may have any value including zero.

More than two low-resolution images may be beamformed for each ultrasound emission, e.g. at least 3, 4, 5, 6, or 10 low-resolution images may be created for each ultrasound emission, thus resulting in at least 3, 4, 5, 6, or 10 high-resolution images. More than two emissions may be used to create the high-resolution images, e.g. at least 3, 4, 5, 8, 16, 32, 64, 128, 192 ultrasound emissions, e.g. each high-resolution image may created by summing at least 3, 4, 5, 8, 16, 32, 64, 128, 192 low-resolution images.

A compounded image may be defined as an image created by processing at least two envelope detected images for reducing the effect of speckle in the envelope detected images. The processing may be performed by summing the envelope detected images and/or by multiplying the envelope detected images by each other and/or by performing another mathematical operation between the at least two envelope detected images. A compounded image may also be created using nonlinear operations, e.g. creating a compounded image comprising for each image point, the maximum or minimum value of the image point in any of the envelope detected images.

Low-resolution images for an emission may differ with respect to the steering angle thus resulting in angular compounding. Additionally or alternatively different frequency intervals may be used to beamform low-resolution images for an emission thus resulting in angular and frequency compounding or frequency compounding. Low-resolution images for an emission may be created with a desired frequency interval by filtering data from an ultrasound emission with a band-pass filter having a passband corresponding to the desired frequency interval, prior to beamforming the data.

Envelope images may also be known as envelope detected images. The process of determining the envelope of a beamformed ultrasound image is commonly performed in ultrasound systems.

Envelope images may be computed by processing for each image line in a high-resolution image processing beamformed or RF data. For narrow band signals with no DC component, the ideal envelope detection can be done by computing the absolute value of the analytic signal. Different realizations of approximations to the ideal envelope detection exist. One solution is to use a single rectifier component or a rectifier bridge followed by a low-pass filter. A precision rectifier is another, more advanced solution where a circuit comprising an operational amplifier is configured to act as a more ideal rectifier for a detection closer to the ideal detection.

In some embodiments, the method comprises transmitting a first ultrasound signal into a physical medium using an ultrasound transducer, receiving a first data signal using said ultrasound transducer said first data signal being indicative of the acoustic properties of said physical medium, creating a first low-resolution image by beamforming data comprising said first data signal using a first set of apodization functions, creating a second low-resolution image by beamforming data comprising said first data signal using a second set of apodization functions,

In some embodiments, the method comprises transmitting a second ultrasound signal into a physical medium using said ultrasound transducer, receiving a second data signal using said ultrasound transducer said second data signal being indicative of the acoustic properties of said physical medium, creating a third low-resolution image by beamforming data comprising said second data signal using a third set of apodization functions, creating a fourth low-resolution image by beamforming data comprising said second data signal using a fourth set of apodization functions.

In some embodiments, the method comprises summing said first and said third low-resolution image creating a first high-resolution image and said second and said fourth low-resolution image creating a second high-resolution image.

In some embodiments, the method comprises computing a first envelope image for said first high-resolution image and a second envelope image for said second high-resolution image, and processing said first envelope image and said second envelope image resulting in a first compounded high-resolution image.

The ultrasound transducer may be a single element transducer or an array transducer comprising a plurality of transducer elements arranged in an array. The ultrasound transducer may be a one-dimensional array transducer or a two-dimensional array transducer. The array transducer may be arranged in a linear array, a convex array or a concave array. The array transducer may be a phased array transducer, where the transducer elements are spaced with a pitch not above half the centre frequency of the ultrasound transducer.

In some embodiments, the ultrasound transducer comprises a plurality of transducer elements, and wherein the first and the second data signal comprise a plurality of RF signals recorded by a subset of said plurality of transducer elements, and where the step of beamforming a low-resolution image comprises, for each point in the low-resolution image, delaying, apodizing, and summing RF signals recorded by said subset of said plurality of transducer elements.

Each set of apodization functions may comprise a plurality of apodization functions, each apodization function comprising a plurality of apodization values. An apodization value is a value used to weight an RF signal recorded by a specific transducer element when beamforming an image point in an image. Each set of apodization functions may comprise a specific apodization function for a given image point in a given low-resolution image, and each apodization function may comprise an apodization value for each transducer element used to beamform a given image point of a given low-resolution image, thus an apodization value for an RF signal recorded by a specific transducer element may be dependent on the specific low-resolution image being beamformed, and the index of the image point in the specific low-resolution image being beamformed.

The subset may comprise at least two transducer elements and/or not all transducer elements and/or all transducer elements of the ultrasound transducer. For a given image point in a low-resolution image corresponding to a spatial position relative to the ultrasound transducer, the beamformed value may be given by the following equation:

$$low\_res {\_im}_{k} ( x , y ) = {\sum }_{n = 1}^{N} {a}_{x , y , k , n} \cdot {S}_{n} ( t - {t}_{f} )$$

where low_res_imₖ(x, y) is the value of the k′th low-resolution image at an image point with index x and y, N is the number of transducer elements of the subset, a_{x,y,k,n} is the apodization value for the image point with index x and y in the k′th low-resolution image for transducer element n, Sₙ(t) is the RF signal recorded by the n′th transducer element of the subset of transducer elements, and t_{f} is a delay value corresponding to an estimated total time of flight for the ultrasound signal transmitted from the transducer to the spatial point corresponding to the image point with index x and y and back to the n′th transducer element. t_{f} may be estimated by estimating the distance the ultrasound signal must travel and combining that estimated distance with an estimated average speed of sound in the physical medium. The above described beamforming method may be known as a kind of delay and sum beamformer, however beamforming may be performed in a variety of ways, e.g. beamforming may be performed in the frequency domain and/or using adaptive algorithms.

In some embodiments, the ultrasound transmissions are low-focused transmissions.

A low-focused ultrasound transmission may be a transmission that does not converge towards a focus point within at least a distance of 2 cm, 3 cm, 5 cm, 10 cm, 20 cm, 30 cm, 40 cm or 50 cm from the ultrasound transducer. The low-focused transmission may an emission from a single ultrasound transducer element or a group of transducer elements, e.g. a defocused ultrasound emission emitted from a group of transducer elements where an ultrasound wavefront is created that converges away from a point positioned at or behind the ultrasound transducer, e.g. by transmitting an ultrasound signal from the group of transducer elements where the ultrasound signal transmitted from the transducer elements of the outer part of the group is delayed relative to the ultrasound signal transmitted from transducer elements of the central part of the group.

By using a low-focused transmission, a more precise estimation of the time of flight for the ultrasound signal from the ultrasound transducer to points in space may be estimated as a more simple transmit wave is generated. Thereby low-resolution image may be beamformed with a more even resolution for all image points. This will further result in high-resolution images with increased resolution.

In some embodiments, said first transmitted ultrasound signal is transmitted with a first subset of ultrasound transducer elements, and said second transmitted ultrasound signal is transmitted with a second subset of ultrasound transducer elements, said first and said second subset of transducer elements differing with at least one transducer element.

In some embodiments, said second transmitted ultrasound signal is directly consecutive of said first transmitted ultrasound signal e.g. there is no ultrasound signals transmitted between said first transmitted ultrasound signal and said second transmitted ultrasound signal.

By combing low-resolution images beamformed from data resulting from ultrasound emissions from different parts of the ultrasound transducer array, high-resolution images may be created with an increased resolution.

In some embodiments, said first data signal is recorded by a third subset of ultrasound transducer elements, and said second data signal is recorded by a fourth subset of transducer elements, said third and said fourth subset differing with at least one transducer element.

By combing low-resolution images beamformed from data recorded by different parts of the ultrasound transducer array, high-resolution images may be created with an increased resolution.

In some embodiment, said first data signal is recorded by a third subset of ultrasound transducer elements, and said second data signal is recorded by a fourth subset of transducer elements, said third and said fourth subset being identical.

In some embodiments, the first set of apodization functions comprises an apodization function for each image point in the first low-resolution image, the second set of apodization functions comprises an apodization function for each image point in the second low-resolution image, the third set of apodization functions comprises an apodization function for each image point in the third low-resolution image, the fourth set of apodization functions comprises an apodization function for each image point in the fourth low-resolution image.

The apodization functions may comprise an apodization value for each transducer element used to beamform a particular image point in a particular low-resolution image, e.g. an apodization function of the first set of apodization functions may comprise an apodization value for each RF signal recorded by each transducer element used to beamform a particular image point in the first low-resolution image, an apodization function of the second set of apodization functions may comprise an apodization value for each RF signal recorded by each transducer element used to beamform a particular image point in the second low-resolution image, etc.

In some embodiments, a set of apodization functions is configured to rotate the resulting point spread function for each image point in a low-resolution image with a predetermined angle so that low-resolution images having point spread functions rotated with different angles may be created.

The point spread function is a measure of the performance of an ultrasound system. It defines the resolution of the system. An estimate of the point spread function of an ultrasound system may be measured by imaging an ultrasound reflector submerged in water e.g. a wire phantom submerged in water. By varying the used apodization functions, the point spread function may be rotated, e.g. by primarily using data recorded by transducer elements positioned in either a first side or a second side of the ultrasound transducer, wherein the point spread function may be rotated in a first or a second direction, respectively.

In some embodiments, a set of apodization functions configured to rotate the resulting point spread function for each image point in a particular low-resolution images with an angle is computed by generating a first line orthogonal to a line having a predetermined angle relative to the ultrasound transducer, wherein the first line intersects a particular image point, placing a first apodization window centred at the particular image point on this first line, generating a second line with an angle relative to the ultrasound transducer equal to the predetermined angle, wherein the second line intersects the centre of emission of the emission used to create the particular low-resolution image. A first part of the apodization value for a particular transducer element, which data are used to beamform the particular image point in the particular low-resolution image, is given as the value of the first apodization window at which the second line and the first apodization window intersects. Similarly, a second part of the apodization value for the particular transducer element, which recorded data are used to beamform the particular image point in the particular low-resolution image, is computed by placing a second apodization window centered at the particular image point on the first line. The second apodization window may be equal to the first apodization window or different from the first apodization window, generating a third line with an angle relative to the ultrasound transducer equal to the predetermined angle, wherein the third line intersects the particular transducer element. The second part of the apodization value is given as the value of the second apodization window at which the third line and the second apodization window intersect. The final apodization value is given by multiplying the first part of the apodization value by the second part of the apodization value. By repeating the above steps for all transducer elements which recorded data are used to beamform the particular image point in the particular low-resolution image, an apodization function for beamforming the particular image point in the particular low-resolution image may be generated. By repeating the above steps for all image points in a particular low-resolution image, a set of apodization functions configured to rotate the resulting point spread function for each image point in the particular low-resolution images with an angle may be generated.

The length of the apodization window(s) may be adjusted as a function of the distance between the image point and the centre of transmission and/or or as a function of the distance between the image point and the receiving transducer element.

Thereby the resolution may be evened out over a range of depths to maintain a constant resolution by realizing a fixed F-number using one or more expanding or contracting apodization window(s).

In some embodiments, the first low-resolution image comprises image data beamformed at a first set of spatial positions, the second low-resolution image comprises image data beamformed a second set of spatial positions, wherein the first and the second set of spatial positions are identical.

In some embodiments, all the low-resolution images comprise image data beamformed at a single set of spatial positions so that all low-resolution images comprise image data from the same spatial positions.

In some embodiments, all the high-resolution images comprises image data from the same set of spatial positions.

All spatial positions may be calculated relative to the position of the ultrasound transducer.

Consequently, compounded images may easily be made without the need or complex re-sampling, e.g. complex scanline conversion.

In some embodiments, the apodization function used to beamform a particular image point in the first low-resolution image has a centre of mass in a first side of the receiving aperture, and the apodization function used to beamform the corresponding image point in the second low-resolution image (e.g. the image point having the same spatial position) has a centre of mass in a second side of the receiving aperture, thus the point spread function of said particular image point has a different orientation in said first and said second low-resolution image.

The receiving aperture for a low-resolution image may be defined as the transducer elements providing the RF signals used to beamform the low-resolution image.

In some embodiments, the apodization function used to beamform a particular image point in the first low-resolution image has a maximum value in a first side of the receiving aperture, and the apodization function used to beamform the corresponding image point in the second low-resolution image (e.g. the image point having the same spatial position) has a maximum value in a second side of the receiving aperture.

In some embodiments, the method further comprises creating a second compounded high-resolution image by combining low-resolution images beamformed using data from at least two ultrasound emissions into at least two high-resolution images, computing at least two envelope images from said at least two high-resolution images and processing said at least two envelope images creating a second compounded high-resolution image, wherein the method further comprises: processing said first and said second compounded high-resolution image creating a combined compounded high-resolution image.

The at least two ultrasound emissions used to create the second compounded high-resolution image may differ with at least one emission from the emissions used to create the first compounded high-resolution image, e.g. at least one emission used to create the second compounded high-resolution image may not be used to create the first compounded high-resolution image. Any method steps used to create the first compounded high-resolution image may equally be used to create the second compounded high-resolution image. The processing of the first and the second compounded high-resolution image may be performed by summing the compounded high-resolution images and/or by multiplying the compounded high-resolution images by each other and/or by performing another mathematical operation between the at least two compounded high-resolution images.

In some embodiments, the method further comprises the step of: providing a user with means to select a contrast/resolution ratio, wherein a compounded image with the selected contrast/resolution ratio is generated.

In some embodiments, the method further comprises the step of: providing a user with means for selecting a contrast/resolution ratio, wherein a compounded image with the selected contrast/resolution ratio is created by:
configuring the sets of apodization functions so that a high contrast/resolution ratio result in an apodization function for a particular image point in a low-resolution image having an effective width that is less than the effective width of the apodization function for said particular image point in said low-resolution image when a low contrast/resolution ratio is selected, and/or
applying a set of weights to a set of low-resolution images forming a high-resolution image, said set of weights being configured to decrease the resolution of the high-resolution images when a high contrast/resolution ratio is selected compared to the resolution of the high-resolution image when a low contrast/resolution ratio is selected.

The effective width of an apodization function may be defined as the length of a particular part of the apodization function comprising a particular percentage of the total energy of the apodization function, e.g. 30%, 40%, 50%, 60% or 80% of the total energy of the apodization function, where the particular part is chosen so that its length is minimized.

The means for selecting a contrast/resolution ratio may be any user input means, e.g. a button, a slider, a voice command system or the like.

The resolution of a high-resolution image may be lowered by applying weights to the low-resolution images summed to give the high-resolution image so that a particular image point in a particular high-resolution image is more influenced by one low-resolution image than another low-resolution image. By primarily using low-resolution images beamformed from ultrasound emissions emitted from a first side of the ultrasound transducer to create a high-resolution image (by applying weighting to the low-resolution image before they are summed into a high-resolution image), high-resolution images may be created having a lower resolution and a point spread function being more rotated. The lower resolution will result in a lower resolution in the compounded ultrasound image; however, the increased rotation will increase the compounding effect increasing the contrast, thus increasing the contrast/resolution ratio.

Contrast influences the ability of the observer to detect small differences in echogenicity of the imaged tissue. A common measure is the contrast-to-noise ratio defined as

$$CNR = \frac{{μ}_{A} - {μ}_{B}}{\sqrt{{σ}_{A}^{2} + {σ}_{B}^{2}}}$$

where μ_{A} is the mean value of the envelope in a region of interest, μ_{B} is the mean value of the envelope of the background. The variances σ_{A}² and σ_{B}² are measures of the variations of the amplitude of the envelope in the region of interest, and in the background, respectively.

Resolution is a measure of the ability of a system to resolve small structures. It is commonly defined as the full-width at half maximum of the point spread function of the imaging system.

In some embodiments, the method further comprises the step of determining automatically a contrast/resolution ratio based on coherence estimation between low-resolution image from different emissions, so that a high contrast/resolution ratio is chosen when a low coherence is detected (indicative of high motion of the ultrasound transducer and/or the tissue being scanned), and a low contrast/resolution ratio is chosen when a high coherence is detected (indicative of low motion of the ultrasound transducer and/or the tissue being scanned), wherein a compounded image with the selected contrast/resolution ratio is created by:
configuring the sets of apodization functions so that a high contrast/resolution ratio result in an apodization function for a particular image point in a low-resolution image having an effective width that is less than the effective width of the apodization function for said particular image point in said low-resolution image when a low contrast/resolution ratio is selected, and/or
applying a set of weights to a set of low-resolution images forming a high-resolution image, said set of weights being configured to decrease the resolution of the high-resolution images when a high contrast/resolution ratio is selected compared to the resolution of the high-resolution image when a low contrast/resolution ratio is selected.

In some embodiments, a first compounded high-resolution image is generated with a first contrast/resolution ratio, and a second compounded high-resolution image is generated with a second contrast/resolution ratio different from the first contrast/resolution ratio, wherein the first compounded high-resolution image and the second compounded high-resolution image are displayed on a display simultaneously.

The first and the second compounded high-resolution image may be generated using the same data.

In some embodiments, for a transmission at least a part of the received data signal is shifted in phase, yielding a phase-shifted data signal, where the data signal and the phase-shifted data signal is combined into a complex data signal where the data signal constitutes the real part and the phase-shifted data signal constitutes the imaginary part, resulting in complex low-resolution images and complex high-resolution images, and wherein an envelope image for a high-resolution image is computed by for an image point calculating:

Eₙ(x,y)=\sqrt{Iₙ(x,y)²+Rₙ(x,y)²}

where Eₙ(x,y) is the value of the n′th envelope image at image point index x,y, Iₙ(x,y) is the imaginary part of the n′th complex high-resolution image at image point index x,y, and Rₙ(x,y) is the real part of the n′th complex high-resolution image at image point index x,y.

All data signals may be shifted in phase, e.g. all RF signals recorded by all receiving transducer elements. The RF signals are shifted in phase on a pr. element basis. The data signals may be shifted approximately 90 degrees in phase. This may be achieved using the Hilbert transformation or approximations to the Hilbert transformation. The complex signals may be beamformed as normal signals, e.g. both the real part and the imaginary part of the complex signals may be delayed and summed resulting in complex beamformed images.

Consequently, envelope images may be created for images beamformed below the Nyquist sampling limit of the RF signals. This greatly lowers the memory and processing requirements of the method decreasing the cost of implementing the method.

According to a second aspect, there is provided an ultrasound system configured to produce compounded ultrasound images by beamforming a first and a second low-resolution image using data from a first ultrasound emission, beamforming a third and a fourth low-resolution image using data from a second ultrasound emission, summing said first and said third low-resolution image creating a first high-resolution image and said second and said fourth low-resolution image creating a second high-resolution image, wherein the ultrasound system further is configured to compute a first envelope image for said first high-resolution image and a second envelope image for said second high-resolution image, and process said first envelope image and said second envelope image creating in a first compounded high-resolution image.

The different aspects of the present invention can be implemented in different ways including the methods of producing compounded ultrasound images and the ultrasound systems described above and in the following, each yielding one or more of the benefits and advantages described in connection with at least one of the aspects described above, and each having one or more preferred embodiments corresponding to the preferred embodiments described in connection with at least one of the aspects described above and/or disclosed in the dependent claims. Furthermore, it will be appreciated that embodiments described in connection with one of the aspects described herein may equally be applied to the other aspects.

### BRIEF DESCRIPTION OF THE DRAWINGS

The above and/or additional objects, features and advantages of the present invention, will be further elucidated by the following illustrative and non-limiting detailed description of embodiments of the present invention, with reference to the appended drawings, wherein:

FIG. 1 shows a flowchart of a method of producing compounded ultrasound images according to an embodiment of the present invention.

FIG. 2 shows a flowchart of a method of producing compounded ultrasound images according to an embodiment of the present invention.

FIG. 3 illustrates a method of producing compounded ultrasound images according to an embodiment of the present invention.

FIG. 4 illustrates a method of producing compounded ultrasound images, according to an embodiment of the present invention.

FIG. 5 shows how an apodization value may be determined according to an embodiment of the present invention.

FIG. 6a-d show examples of apodization functions generated according to an embodiment of the present invention.

### DETAILED DESCRIPTION

In the following description, reference is made to the accompanying figures, which show by way of illustration how the invention may be practiced.

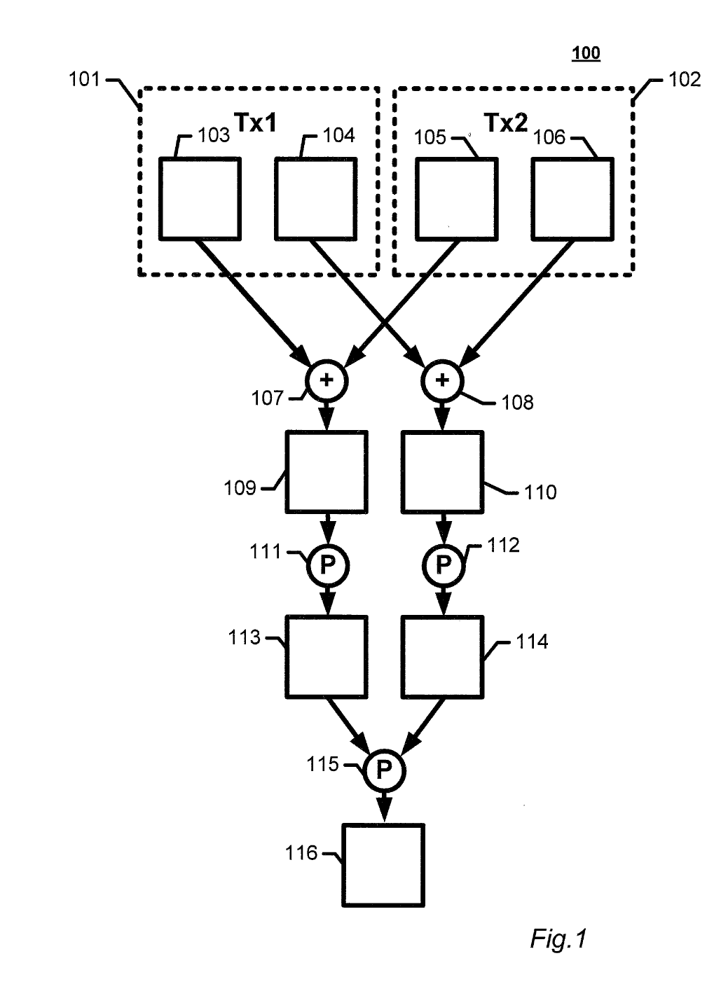

FIG. 1 shows a schematic drawing of a method of producing compounded ultrasound images 100 according to an embodiment of the present invention. A first low-resolution image 103 and a second low-resolution image 104 are beamformed using data from a first ultrasound transmission 101. A third low-resolution image 105 and a fourth low-resolution image 106 are beamformed using data from a second ultrasound emission 102. Each low-resolution image comprises image data beamformed at identical spatial positions relative to the ultrasound transducer so that they may be easily processed together without the need for complex re-sampling. The first and the third low-resolution image 103 105 are added 107 forming a first high-resolution image 109. Correspondingly, the second and the fourth low-resolution image 104 106 are added 108 forming a second high-resolution image 110. The first high-resolution image 109 is processed 111 to obtain a first envelope image 113, and the second high-resolution image 110 is processed 112 to obtain a second envelope image 114. The first and the second envelope image 113 114 are processed together 115 to obtain a first compounded high-resolution image 116 having a high resolution in all image points and a reduced speckle effect.

Beamforming of low-resolution images may be done directly on the RF signals recorded by a plurality of transducer elements making up the receiving aperture. Alternatively, beamforming may be done on the complex analytical counterpart of the RF signals which has both in-phase and quadrature components. The RF data signals are shifted in phase yielding phase-shifted data signals, where the data signals and the phase-shifted data signals are combined into complex data signals where the data signals constitute the real part and the phase-shifted data signals constitute the imaginary part. Finding the imaginary part of the complex analytical counterpart of the RF signals can be done by computing the Hilbert Transform of the RF signals. When the process of beamforming uses complex signals, the result is complex low-resolution images and complex high-resolution images from which envelope detection is computed.

Every low-resolution image consists of a number of points. If beamforming is based on RF signals as input signals, the points may be placed in lines making up image lines and where the distance between image lines and the distance between points in an image line are determined by the Nyquist sampling criterion for the RF signals. Working with complex in-phase and quadrature data makes it possible to beamform only those points in which the envelope detected value is needed. The bandwidth of the detected signal is much lower than the RF signals, and the points making up the low-resolution images may be placed in a less dense grid than when working with RF signals in the beamformer.

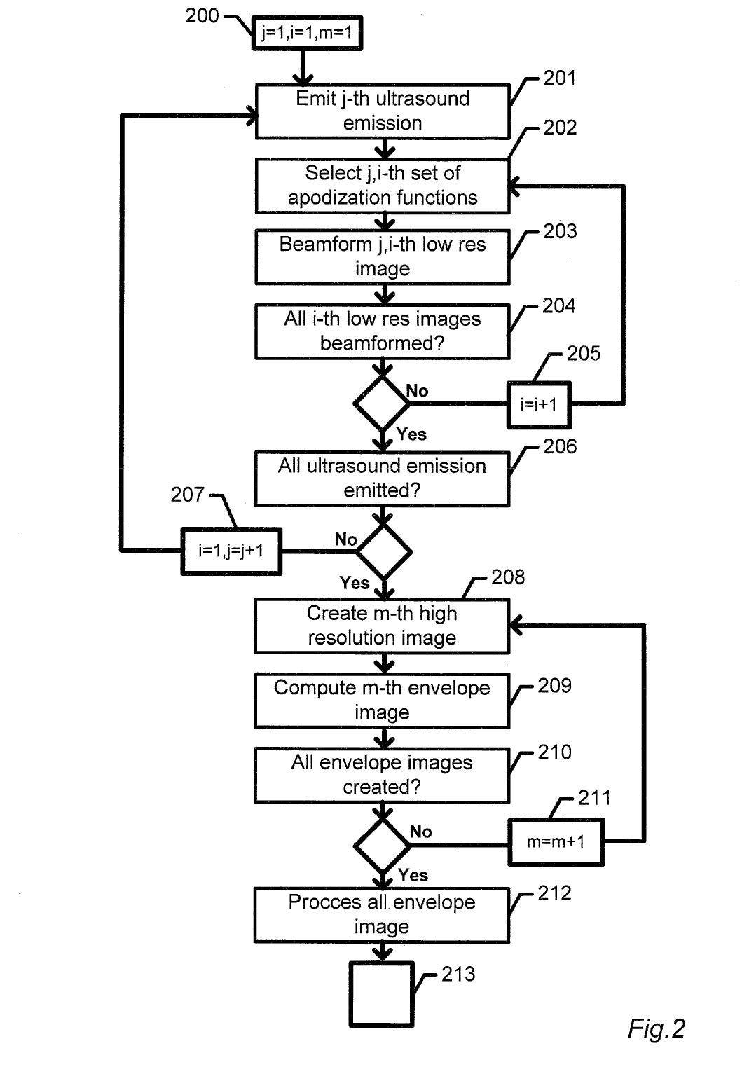

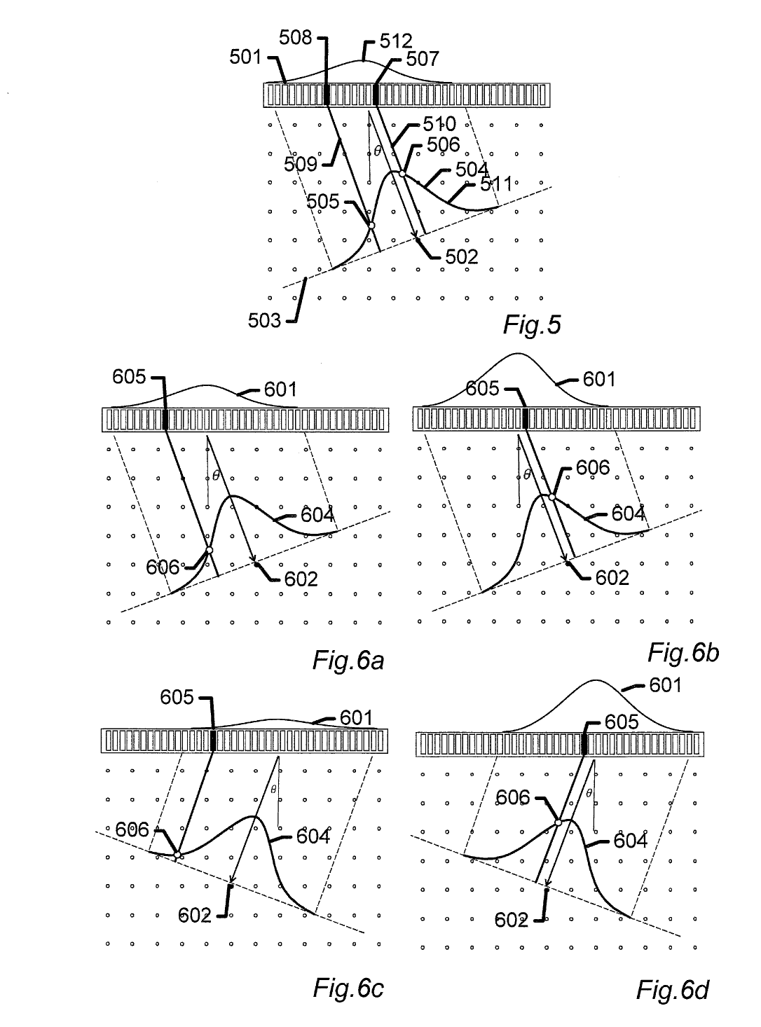

FIG. 2 shows a flowchart of a method of producing compounded ultrasound image according to an embodiment of the present invention. First, a first ultrasound signal is emitted from an ultrasound transducer in step 201. Using a receiving aperture of the ultrasound transducer, the reflected echoes of the emitted ultrasound signal are recorded resulting in a first data signal, e.g. a plurality of RF signals one for each transducer element of the receiving aperture. Then, to beamform a particular low-resolution image, a specific set of apodization functions is chosen in step 202 dependent on the specific low-resolution image. In step 203 the chosen set of apodization functions is used to beamform a specific low-resolution image by processing the first data signal. In step 204 the method determines if all low-resolution images for the specific ultrasound emission have been beamformed. If the method determines that not all low-resolution images have been beamformed, it returns to step 202 and selects a new set of apodization functions and uses the set of apodization functions to beamform a new low-resolution image using the first data signal. The different low-resolution images for a specific ultrasound emission may differ with respect to the orientation of the point spread function. The point spread function may be rotated by primarily using data recorded by a particular side of the receiving aperture when beamforming a low-resolution image. This may be achieved by using specially designed apodization functions, e.g. as described in relation to FIG. 5. On the other hand, if the method determines that all low-resolution images for the specific ultrasound emission have been beamformed, it proceeds to step 206 where the method determines if all ultrasound emissions have been emitted. If the method determines that not al ultrasound emissions have been emitted, it returns to step 201, emits a new ultrasound emission records a new data set, and beamforms a number of low-resolution images using different sets of apodization functions. If the method, however, determines that all ultrasound emissions have been emitted, it proceeds to step 208 where a high-resolution image is generated by summing a number of low-resolution images, e.g. summing the first low-resolution image beamformed for each ultrasound emission. By processing the generated high-resolution image, the method creates an envelope image, in step 209. In step 210, the method determines if all envelope images have been created. If the method determines that not all envelope images have been created, it returns to step 208 where a new high-resolution image is created by summing low-resolution images, e.g. summing the second low-resolution image beamformed for each ultrasound emission. However, if the method determines that all envelope images have been created, it continues to step 212 where the envelope images are processed together, as described above or below, resulting in a compounded high-resolution image 213.

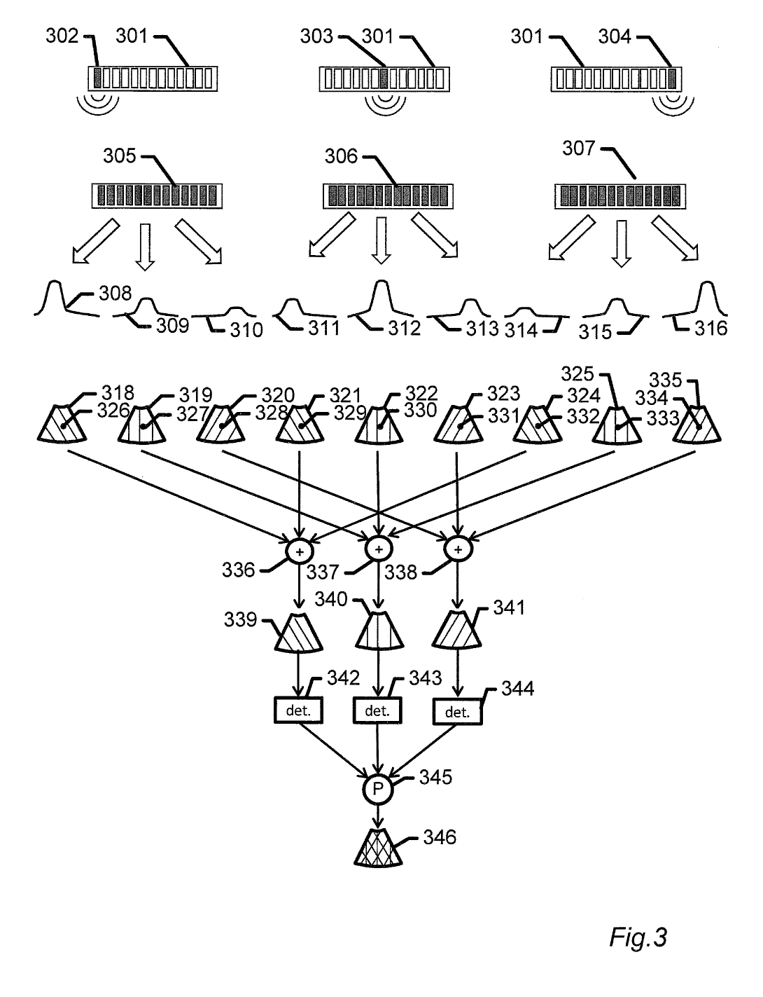

FIG. 3 illustrates a method of producing compounded ultrasound images according to an embodiment of the present invention. The acquisition is performed by use of an array of ultrasound elements 301. First a defocused wave is transmitted with a center of emission 302. The defocused wave may be transmitted by a single transducer element or a group of transducer elements where the ultrasound signal transmitted from the transducer elements of the outer part of the group is delayed relative to the ultrasound signal transmitted from transducer elements of the central part of the group. The wave propagates in the whole region of investigation. The back scattered signal is recorded using a receive aperture 305. Recorded channel data are used to generate three low-resolution images 318 319 320 using delay-and-sum beamforming. The delays and apodization coefficients vary from image point to image point in the image. Shown are three apodization functions 308 309 310, used to beamform the centre image point 326 327 328 of the three low-resolution image 318 319 320, respectively. The apodization function 308 has a centre of mass in the left part of the receiving aperture and as a result the point spread function for the centre point 326 in the low-resolution image 318 is rotated to the left with respect to the receiving aperture. The apodization function 309 has a centre of mass in the centre of the receiving aperture. Consequently the point spread function for the centre point 327 in the low-resolution image 319 is not rotated with respect to the receiving aperture. The apodization function 310 has a centre of mass in the right part of the receiving aperture. As a result the point spread function for the centre point 328 in the low-resolution image 320 is rotated to the right with respect to the receiving aperture. It can further be observed that the height of the apodization function 308 is higher than the apodization functions 309 310, and the height of the apodization function 309 is higher than the height of the apodization function 310. The apodization functions for the majority of the remaining points in each of the low-resolution images 318 319 320 may be similar to the apodization function of the centre point, thus the low-resolution image 318 is angled to the left, the low-resolution image 319 is not angled, and the low-resolution image 320 is angled to the right. As the height of the apodization functions used to beamform the low-resolution image 318 are higher than the height of the apodization functions used to beamform the low-resolution images 319 320, the low-resolution image 318 generally comprises larger values (possibly positive and negative) compared to the low-resolution images 319 320. Correspondingly, as the heights of the apodization functions used to beamform the low-resolution image 319 are higher than the heights of the apodization functions used to beamform the low-resolution image 320, the low-resolution image 319 generally comprises larger (possibly positive and negative) values compared to the low-resolution images 320.

The acquisition using a defocused transmission is repeated with another center of emission 303. The wave propagates in the whole region of interest. The back scattered signal is recorded using a receive aperture 306. Recorded channel data are used to generate three low-resolution images 321, 322 and 323 using delay-and-sum beamforming. The delays and apodization functions vary from image point to image point in the image. Shown are three apodization functions 311 312 313 used to beamform the centre image point 329, 330 331 of the three low-resolution images 321 322 323, respectively. The apodization function 311 has a centre of mass in the left part of the receiving aperture, and as a result the point spread function for the centre point 329 in the low-resolution image 321 is rotated to the left with respect to the receiving aperture. The apodization function 312 has a centre of mass in the centre of the receiving aperture. Consequently the point spread function for the centre point 330 in the low-resolution image 322 is not rotated with respect to the receiving aperture. The apodization function 313 has a centre of mass in the right part of the receiving aperture. As a result the point spread function for the centre point 331 in the low-resolution image 323 is rotated to the right with respect to the receiving aperture. It can further be observed that the height of the apodization function 312 is higher than the apodization functions 311 313, and the heights of the apodization functions 311 313 are the same. The apodization functions for the majority of the remaining points in each of the low-resolution images 321 322 323 may be similar to the apodization function of the centre point, thus the low-resolution image 321 is angled to the left, the low-resolution image 322 is not angled, and the low-resolution image 323 is angled to the right. As the heights of the apodization functions used to beamform the low-resolution image 322 are higher than the heights of the apodization functions used to beamform the low-resolution images 321 323, the low-resolution image 322 generally comprises larger values (possibly positive and negative) compared to the low-resolution images 321 322. Correspondingly as the heights of the apodization functions used to beamform the low-resolution images 321 323 are the same, the low-resolution images 321 323 generally comprise values of equal size.

The acquisition using a defocused transmission is repeated with another center of emission 304. The wave propagates in the whole region of interest. The back scattered signal is recorded using a receive aperture 307. Recorded channel data are used to generate three low-resolution images 324 325 335 using delay-and-sum beamforming. The delays and apodization functions vary from image point to image point in the images. Shown are three apodization functions 314 315 316 used to beamform the centre image point 332, 333 334 of the three low-resolution images 324 325 335, respectively. The apodization function 314 has a centre of mass in the left part of the receiving aperture, and as a result the point spread function for the centre point 332 in the low-resolution image 324 is rotated to the left with respect to the receiving aperture. The apodization function 315 has a centre of mass in the centre of the receiving aperture. Consequently the point spread function for the centre point 333 in the low-resolution image 325 is not rotated with respect to the receiving aperture. The apodization function 316 has a centre of mass in the right part of the receiving aperture. As a result the point spread function for the centre point 334 in the low-resolution image 335 is rotated to the right with respect to the receiving aperture. It can further be observed that the height of the apodization function 316 is higher than the apodization functions 314 315, and the height of the apodization function 315 is higher than the height of the apodization function 314. The apodization functions for the majority of the remaining points in each of the low-resolution images 324 325 335 may be similar to the apodization function of the centre point. Thus the low-resolution image 324 is angled to the left, the low-resolution image 325 is not angled and the low-resolution image 335 is angled to the right. As the height of the apodization functions used to beamform the low-resolution image 335 are higher than the height of the apodization functions used to beamform the low-resolution images 325 324, the low-resolution image 335 generally comprises larger values (possibly positive and negative) compared to the low-resolution images 324 325. Correspondingly as the height of the apodization functions used to beamform the low-resolution image 325 are higher than the height of the apodization functions used to beamform the low-resolution image 324, the low-resolution image 325 generally comprises larger (possibly positive and negative) values compared to the low-resolution images 324.

The low-resolution images 318 321 324 angled to the left by the used apodization functions, e.g. apodization functions 308 311 314, are summed 336 into high-resolution image 339 correspondingly angled to the left. The heights of the apodization functions used to beamform the low-resolution image 318 are generally higher than the heights of the apodization functions used to beamform the low-resolution images 321 324. The low-resolution image 318 therefore generally comprises larger values (possibly positive and negative) compared to the low-resolution images 321 324. Correspondingly, the heights of the apodization functions used to beamform the low-resolution image 321 are generally higher than the heights of the apodization functions used to beamform the low-resolution image 324. The low-resolution image 321 therefore generally comprises larger values (possibly positive and negative) compared to the low-resolution image 324. As a result, the high-resolution image 339 is more influenced by the low-resolution images beamformed from emissions from the left part of the array. Thereby, a transmit apodization is synthesized, further rotating the point spread functions of the image points in the high-resolution image 339 to the left.

The low-resolution images 319 322 325 not angled by the used apodization functions, e.g. apodization functions 309 312 315, are summed 337 into high-resolution image 340 correspondingly not angled. The heights of the apodization functions used to beamform the low-resolution image 322 are generally higher than the heights of the apodization functions used to beamform the low-resolution images 319 325. The low-resolution image 322 therefore generally comprises larger values (possibly positive and negative) compared to the low-resolution images 319 325. As a result, the high-resolution image 340 is more influenced by the low-resolution images beamformed from emissions from the centre part of the array. Thereby a transmit apodization is synthesized not rotating the point spread functions of the image points in the high-resolution image 340.

The low-resolution images 320 323 335 angled to the right by the used apodization functions, e.g. apodization functions 310 313 316, are summed 338 into high-resolution image 341 correspondingly angled to the right. The height of the apodization functions used to beamform the low-resolution image 335 are generally higher than the height of the apodization functions used to beamform the low-resolution images 320 323. The low-resolution image 335 therefore generally comprises larger values (possibly positive and negative) compared to the low-resolution images 320 323. Correspondingly, the heights of the apodization functions used to beamform the low-resolution image 323 are generally higher than the heights of the apodization functions used to beamform the low-resolution image 320. The low-resolution image 323 therefore generally comprises larger values (possibly positive and negative) compared to the low-resolution image 320. As a result the high-resolution image 341 is more influenced by the low-resolution images beamformed from emissions from the right part of the array. Thereby a transmit apodization is synthesized, further rotating the point spread functions of the image points in the high-resolution image 341 to the right.

The envelope of each image lines of the high-resolution image angled to the left 339 is computed 342 to produce an envelope image correspondingly angled to the left, the envelope of each image lines of the high-resolution image not angled 340 being computed 343 to produce an envelope image correspondingly not angled, and the envelope of each image lines of the high-resolution image angled to the right 341 being computed 344 to produce an envelope image 344 correspondingly angled to right. By processing 345 the envelope images at the output of 342 343 344, e.g. summing them, multiplying them by each other, or performing other linear or nonlinear operations, a compounded high-resolution image 346 is created having a high resolution in all image points and being suppressed for speckle. It should be understood that more or less all ultrasound emissions may be used, e.g. at least 2, 4, 5, 8, 16, 32, 64, 128, or 192 ultrasound emissions and/or more or less low-resolution images, e.g. at least 2, 4, 5, 6, 8, 10, or 20 low-resolution images.

FIG. 5 shows how an apodization value used to weight the output of a particular receive transducer element 507 when beamforming a particular image point 502 in a particular low-resolution image may be generated according to an embodiment of the present invention. Shown is a transducer array 501 comprising a plurality of transducer elements. First, a first line 503 is generated orthogonal to a line having predetermined angle θ relative to the ultrasound transducer, wherein the angle θ is related to a desired angling of the point spread function of the particular low-resolution image. The first line 503 is positioned so that it intersects the particular image point 502. On the first line 503 is placed a first apodization window 504, in this example a Hanning window; however, other window functions may be used, e.g. a boxcar or Hamming etc. The first apodization window 504 is centred around the particular image point 502. To calculate a first part of the apodization value 505 for a particular receive transducer element 507, which data are used for beamforming, the particular image point 502 of the particular low-resolution image, the first apodization window 504 is projected onto the centre of emission 508 for the particular emission used to generate the particular low-resolution image. This is done by generating a second line 509 which is orthogonal to the first line 503 and intersecting the centre of emission 508. The first part of the apodization value 505 is given as the value of the first apodization window 504 at which the second line 509 and the first apodization window 504 intersect.

Similarly, a second part of the apodization value 506 for the particular transducer element 507, which recorded data are used to beamform the particular image point 502 in the particular low-resolution image, is computed by placing a second apodization window 511 centered at the particular image point 502 on the first line 503. The second apodization window 511 is, in this embodiment, equal to the first apodization window 504; however, in other embodiments they may differ. To calculate the second part of the apodization value for a particular receive transducer element 507, which data are used for beamforming the particular image point 502 of the particular low-resolution image, the second apodization window 511 is projected onto the particular receive transducer element 507. This is done by generating a third line 510 which is orthogonal to the first line 503, wherein the third line intersects the particular receive transducer element 507. The second part of the apodization value 506 is given as the value of the second apodization window 511 at which the third line 510 and the second apodization window 511 intersect. The final apodization value is given by multiplying the first part of the apodization value 505 by the second part of the apodization value 506. By repeating the above steps for all transducer elements, which recorded data are used to beamform the particular image point 502 in the particular low-resolution image, an apodization function 512 for beamforming the particular image point 502 in the particular low-resolution image may be generated. By repeating the above steps for all image points in a particular low-resolution image, a set of apodization functions configured to rotate the resulting point spread function for each image point in the particular low-resolution images with an angle may be generated.

FIG. 6a-d show examples of apodization functions for image points generated using the method described in relation to FIG. 5.

FIG. 6a shows the resulting apodization function 601 for image point 602 in a particular low-resolution image when it is desired to rotate the resulting point spread function to the left. The transducer element 605 shows the centre of emission for the ultrasound emission used to generate the particular low-resolution image. The resulting apodization function 601 is generated by projecting the apodization window 604 onto the transducer elements used to beamform the image point 602 and scaling the projected preliminary apodization function with the value 606 corresponding to the value of the apodization window 604 projected onto the centre of emission 605 for the ultrasound emission used to generate the particular low-resolution image.

FIG. 6b shows the resulting apodization function 601 for image point 602 in a particular low-resolution image when it is desired to rotate the resulting point spread function to the left. The transducer element 605 shows the centre of emission for the ultrasound emission used to generate the particular low-resolution image. The resulting apodization function 601 is generated by projecting the apodization window 604 onto the transducer elements used to beamform the image point 602 and scaling the projected apodization window with the value 606 corresponding to the value of the apodization window 604 projected onto the centre of emission 605 for the ultrasound emission used to generate the particular low-resolution image.

FIG. 6c shows the resulting apodization function 601 for image point 602 in a particular low-resolution image when it is desired to rotate the resulting point spread function to the right. The transducer element 605 shows the centre of emission for the ultrasound emission used to generate the particular low-resolution image. The resulting apodization function 601 is generated by projecting the apodization window 604 onto the transducer elements used to beamform the image point 602 and scaling the projected apodization window with the value 606 corresponding to the value of the apodization window 604 projected onto the centre of emission 605 for the ultrasound emission used to generate the particular low-resolution image.

FIG. 6d shows the resulting apodization function 601 for image point 602 in a particular low-resolution image when it is desired to rotate the resulting point spread function to the right. The transducer element 605 shows the centre of emission for the ultrasound emission used to generate the particular low-resolution image. The resulting apodization function 601 is generated by projecting the apodization window 604 onto the transducer elements used to beamform the image point 602 and scaling the projected preliminary apodization function with the value 606 corresponding to the value of the preliminary apodization window 604 projected onto the centre of emission 605 for the ultrasound emission used to generate the particular low-resolution image.

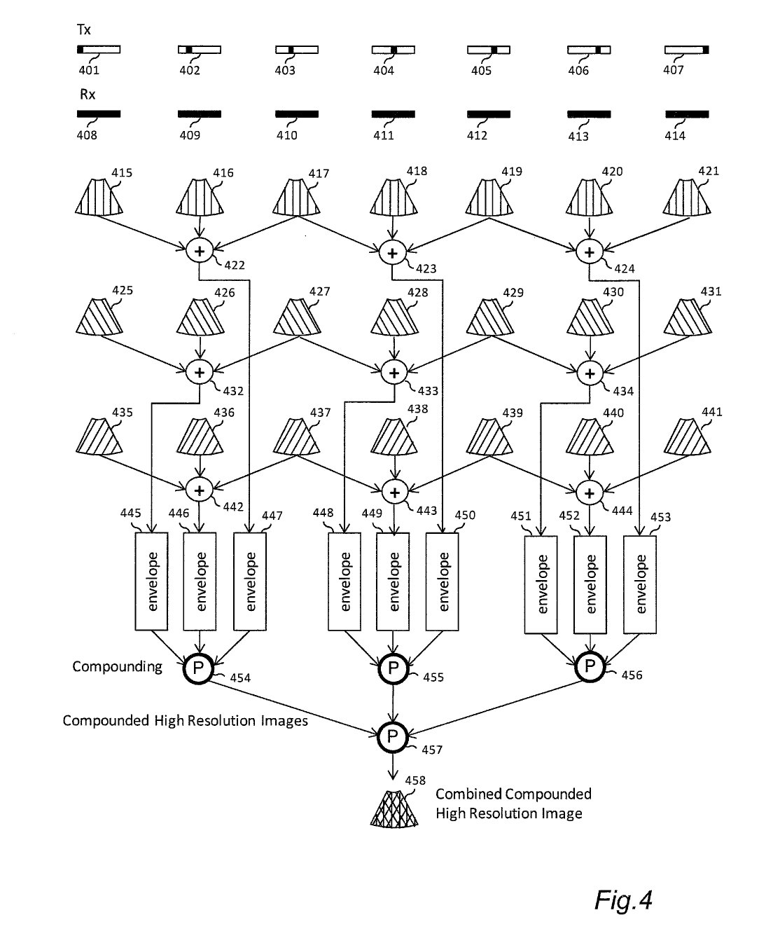

FIG. 4 shows a method of producing a combined compounded high-resolution image according to an embodiment of the present invention. A number of sequential emissions (Ne) 401-407 are used to process a single combined compounded high-resolution image 458. In this embodiment, Ne is 7, but it may be higher or lower. Each of the Ne emissions may be a focused emission, an unfocused emission, a plane wave emission, a defocused emission, or a spherical wave emission from a single element. The transmit apertures 401-407 indicate the origins of the different emissions. Ne different origins are used for the Ne emissions and these Ne origins may be distributed equally over the entire transducer.

For each emission, the ultrasound transducers comprising a plurality of transducer elements are used to record a set of RF channel data from all transducer elements as in this embodiment or from a subset of the plurality of transducer elements if the number of receiving channels in the ultrasound system is limited to a number less than the number of transducer elements.

For the emission 401, a set of RF signals is recorded by the plurality of transducer elements making up the receiving aperture 408. These data are used for beamforming a number of low-resolution images 415, 425, 435, by for each point in the resolution image delaying, apodizing, and summing RF signals recorded by the plurality of transducer elements making up the receiving aperture 408. The number of low-resolution images being beamformed for each emission is 3 in this embodiment, but it may be higher (or lower) for a larger suppression of speckle thus improving contrast.

Beamforming of low-resolution images can be done directly on the RF signals recorded by the plurality of transducer elements making up the receiving aperture 408. Beamforming can also be done on the complex analytical counterpart of the RF signals which has both in-phase and quadrature components. The RF data signals are shifted in phase yielding phase-shifted data signals, where the data signals and the phase-shifted data signals are combined into complex data signals where the data signals constitute the real part and the phase-shifted data signals constitute the imaginary part. Finding the imaginary part of the complex analytical counterpart of the RF signals can be done by computing the Hilbert Transform of the RF signals. When the process of beamforming uses complex signals, the result is complex low-resolution images and complex high-resolution images from which envelope detection is computed.

Every low-resolution image consists of a number of points. If beamforming is based on RF signals as input signals, the points may be placed in lines making up image lines and, where the distance between image lines and the distance between points in an image line are determined by the Nyquist sampling criterion for the RF signals. Working with complex in-phase and quadrature data makes it possible to beamform only those points in which the envelope detected value is needed. As the bandwidth of the detected signal may be lower than the bandwidth of the RF signals, the points making up the low-resolution images may be placed in a less dense grid than when working with RF signals in the beamformer. In the embodiment described, beamforming is done on complex data and every low-resolution image beamformed from every emission comprises beamformed samples at identical spatial positions. However, beamforming may also be done directly on RF signals.

Each of the low-resolution images 415, 425, 435 is beamformed using the same set of delay profiles. A set of delay profiles comprises a delay profile for each point in the low-resolution image. A delay profile comprises a set of delay values, one delay value for each of the plurality of transducer elements making up the receiving aperture 408. A delay value for a given receiving element corresponds to an estimated total time of flight for the ultrasound signal transmitted from the transmit aperture origin 401 to the spatial point corresponding to a given point in the low-resolution image and back to the given receiving element. The time of flight may be estimated by estimating the distance the ultrasound signal must travel and dividing that estimated distance by an estimated average speed of sound in the physical medium. This beamforming process of the embodiment may be characterized as time domain delay and sum beamforming. Other beamforming techniques can be utilized here as a replacement of time domain delay and sum beamforming, such as frequency domain delay and sum beamforming, or adaptive beamforming algorithms.

The low-resolution images 415, 425, 435 are beamformed using different sets of apodization functions emulating different steering directions. Each set of apodization functions may comprise a plurality of apodization functions, and in one embodiment there is one apodization function for each point in the low-resolution image, and wherein each apodization function comprises a plurality of apodization values and, in one embodiment, one value for each of the plurality of transducer elements making up the receiving aperture 408. An apodization value is a value used to weight an RF signal when beamforming a given point in a given low-resolution image for a given steering direction; thus, an apodization value for an RF signal recorded by a specific transducer element may be dependent on the specific low-resolution image being beamformed and the index of the image point in the specific low-resolution image being beamformed. An apodization value may comprise a first part determined by the centre of emission for the ultrasound emission and a desired angling of the point spread function, and a second part determined by the position of the receiving transducer element and the desired angling of the point spread function. The apodization value may effectively be the product of the two parts.

The principle of computation of the apodization value is illustrated in FIG. 5. The steps of beamforming a low-resolution image thus comprise for each point in the low-resolution image delaying, apodizing, and summing RF signals recorded by the plurality of transducer elements.

The beamformed low-resolution images 415, 425, 435 are stored in a number of buffers, the number being equal to the number of steering angles or a number being equal to the total number of low-resolution images that are created in the process of creating a combined compounded high-resolution image 458, or a number in between. If the number of buffers is equal to the number of steering angles, each buffer serves as a storage for accumulated low-resolution images from a given steering angle. If the number of buffers is larger, it yields a more flexible system, but requires a larger memory for data storage.

For the emission 402, a set of RF signals is recorded by the plurality of transducer elements making up the receiving aperture 409. These data are used for beamforming a number of low-resolution images 416, 426, 436, and it comprises, for each point in the low-resolution image, delaying, apodizing, and summing RF signals recorded by the plurality of transducer elements making up the receiving aperture 409.

The beamformed low-resolution images 416, 426, 436 from the emission 402 are beamformed using different sets of apodization functions emulating different steering directions and are stored in buffers.

For the emission 403 a set of RF signals is recorded by the plurality of transducer elements making up the receiving aperture 410. These data are used for beamforming a number of low-resolution images 417, 427, 437 by for each point in the low-resolution image, delaying, apodizing, and summing RF signals recorded by the plurality of transducer elements making up the receiving aperture 410.

The beamformed low-resolution images 417, 427, 437 from the emission 403 are beamformed using different sets of apodization functions emulating different steering directions and are stored in buffers.

The low-resolution images 415, 416, 417 comprise beamformed image points with the same spatial positions. They are beamformed with a first steering direction using RF data acquired from 3 different emission with 3 different transmit apertures 401, 402, 403 having 3 different positions of the transmit origins. Coherently adding the complex in-phase and quadrature low-resolution images 415, 416, 417 in the adder 422 yields a high-resolution image with a first steering direction and with a synthesized transmit aperture that is larger than the individual transmit apertures 401, 402, 403 thus yielding an improved resolution. Adding the low-resolution images 415, 416, 417 in the adder 422 may comprise a weighting of the low-resolution images 415, 416, 417 before they are added in 422.

Similarly the low-resolution images 425, 426, 427 comprise beamformed image points with the same spatial positions, and further with the same spatial positions as the low-resolution images 415, 416, 417. The low-resolution images 425, 426, 427 are beamformed with a second steering direction using RF data acquired from 3 different emission with 3 different transmit apertures 401, 402, 403 having 3 different positions of the transmit origins. Coherently adding the complex in-phase and quadrature low-resolution images 425, 426, 427 in the adder 432 yields a high-resolution image with a second steering direction and with a synthesized transmit aperture that is larger than the individual transmit apertures 401, 402, 403 thus yielding an improved resolution. Adding the low-resolution images 425, 426, 427 in the adder 432 may comprise a weighting of the low-resolution images 425, 426, 427 before they are added in 432.

Similarly the low-resolution images 435, 436, 437 comprise beamformed image points with the same spatial positions and further with the same spatial positions as the low-resolution images 415, 416, 417, 425, 426, 427. The low-resolution images 435, 436, 437 are beamformed with a third steering direction using RF data acquired from 3 different emission with 3 different transmit apertures 401, 402, 403 having 3 different positions of the transmit origins. Coherently adding the complex in-phase and quadrature low-resolution images 435, 436, 437 in the adder 442 yields a high-resolution image with a third steering direction and with a synthesized transmit aperture that is larger than the individual transmit apertures 401, 402, 403 thus yielding an improved resolution. Adding the low-resolution images 435, 436, 437 in the adder 442 may comprise a weighting of the low-resolution images 435, 436, 437 before they are added in 442.

The number of low-resolution images being added in the adders 422, 432, 442 is 3 in this embodiment. This number may be higher for an extended synthesized aperture yielding an improved resolution. The number is determined by the desired contrast/resolution ratio and may depend on whether the image medium is stationary or moving. For an image medium that moves, it may be desirable to keep this number low to ensure a coherent summation.

Consider a high-resolution image with a given steering direction comprising a grid of beamformed points created from beamforming low-resolution images using complex in-phase and quadrature RF signals and finally adding low-resolution images. The delay and apodization functions applied in the beamforming process are configured such that the point spread function at a given point in the high-resolution image corresponds to the point spread function at the same spatial position of an image constructed by having steered and focused emissions and steered beams beamformed in the direction of the said steering direction.

The complex in-phase and quadrature high-resolution images at the output of the adders 422, 432, 442 are envelope detected in 445, 446, 447. The detection is computed as the magnitude of the complex image.

The envelope detected high-resolution images at the output of the envelope detectors 445, 446, 447 comprises beamformed image points with the same spatial positions, but with a first, a second, and a third steering direction. The envelope detected high-resolution images are processed together, e.g. by adding these images and creating a compounded high-resolution image comprising, for each image point, e.g. an addition of image points constructed as if they were observed from a first, and a second, and a third observation angle suppressing the speckle and improving the contrast of the compounded high-resolution image.

The above process of constructing a first compounded high-resolution image based on the emissions 401, 402, 403 is repeated for the emissions 403, 404, 405 constructing the second compounded high-resolution image, and is repeated for the emissions 405, 406, 407 constructing the third compounded high-resolution image. In this embodiment emission 403 is used for constructing both the first and the second compounded high-resolution image, and emission 405 is used for constructing both the second and the third compounded high-resolution image; however, in other embodiments there may be no overlap between the emissions used to create the different compounded high-resolution images, e.g. emissions 401 402 403 may be used to create a first compounded high-resolution image, and emissions 404 405 406 may be used to create a second compounded high-resolution image, or there may be an increased overlap, e.g. emissions 401 402 403 may be used to create a first compounded high-resolution image and emission 402 403 404 may be used to create a second compounded high-resolution image.

The final combined compounded high-resolution image 458 is constructed by processing the compounded high-resolution images, e.g. by adding them. The compounded high-resolution image may be weighted before they are processed, e.g. they may be weighted before they are added.

Although some embodiments have been described and shown in detail, the invention is not restricted to them, but may also be embodied in other ways within the scope of the subject matter defined in the following claims. In particular, it is to be understood that other embodiments may be utilised and structural and functional modifications may be made without departing from the scope of the present invention.

In device claims enumerating several means, several of these means can be embodied by one and the same item of hardware. The mere fact that certain measures are recited in mutually different dependent claims or described in different embodiments does not indicate that a combination of these measures cannot be used to advantage.

It should be emphasized that the term “comprises/comprising” when used in this specification is taken to specify the presence of stated features, integers, steps or components but does not preclude the presence or addition of one or more other features, integers, steps, components or groups thereof.

1. A method for producing compounded ultrasound images, the method comprising:
beamforming a first and a second low-resolution image using data from a first ultrasound emission,
beamforming a third and a fourth low-resolution image using data from a second ultrasound emission,
summing said first and said third low-resolution image creating a first high-resolution image and said second and said fourth low-resolution image creating a second high-resolution image,
computing a first envelope image for said first high-resolution image and a second envelope image for said second high-resolution image, and
processing said first envelope image and said second envelope image creating in a first compounded high-resolution image.

2. A method according to claim 1, where the method further comprises:
transmitting a first ultrasound signal into a physical medium using an ultrasound transducer,
receiving a first data signal using said ultrasound transducer said first data signal being indicative of the acoustic properties of said physical medium,
creating a first low-resolution image by beamforming data comprising said first data signal using a first set of apodization functions, and
creating a second low-resolution image by beamforming data comprising said first data signal using a second set of apodization functions.

3. A method according to claim 1, where the method further comprises:
transmitting a second ultrasound signal into a physical medium using said ultrasound transducer,
receiving a second data signal using said ultrasound transducer said second data signal being indicative of the acoustic properties of said physical medium,
creating a third low-resolution image by beamforming data comprising said second data signal using a third set of apodization functions, and
creating a fourth low-resolution image by beamforming data comprising said second data signal using a fourth set of apodization functions.

4. A method according to claim 1, where the method further comprises:
summing said first and said third low-resolution image creating a first high-resolution image and said second and said fourth low-resolution image,
creating a second high-resolution image,
computing a first envelope image for said first high-resolution image and a second envelope image for said second high-resolution image, and
processing said first envelope image and said second envelope image resulting in a first compounded high-resolution image.

5. A method according to claim 1, where the ultrasound transducer comprises a plurality of transducer elements, and wherein the first and the second data signal comprise a plurality of RF signals recorded by a subset of said plurality of transducer elements, and where the step of beamforming a low-resolution image comprises, for each point in the low-resolution image, delaying, apodizing, and summing RF signals recorded by said subset of said plurality of transducer elements.

6. A method according to claim 1, where a set of apodization functions is configured to rotate the resulting point spread function for each image point in a low-resolution image with a predetermined angle so that low-resolution images having point spread functions rotated with different angles may be created.

7. A method according to claim 1, where the method further comprises:
creating a second compounded high-resolution image by combining low-resolution images beamformed using data from at least two ultrasound emissions into at least two high-resolution images,
computing at least two envelope images from said at least two high-resolution images,
processing said at least two envelope images creating a second compounded high-resolution image, and
processing said first and said second compounded high-resolution image creating a combined compounded high-resolution image.

8. A method according to claim 1, where the method further comprises the step of: providing a user with means to select a contrast/resolution ratio, wherein a compounded image with the selected contrast/resolution ratio is generated.

9. A method according to claim 1, where for a transmission at least a part of the received data signal is shifted in phase, yielding a phase-shifted data signal, where the data signal and the phase-shifted data signal is combined into a complex data signal where the data signal constitutes the real part and the phase-shifted data signal constitutes the imaginary part, resulting in complex low-resolution images and complex high-resolution images, and wherein an envelope image for a high-resolution image is computed by for an image point calculating:
Eₙ(x,y)=\sqrt{Iₙ(x,y)²+Rₙ(x,y)²}
where Eₙ(x,y) is the value of the n′th envelope image at image point index x,y, lₙ(x,y) is the imaginary part of the n′th complex high-resolution image at image point index x,y, and Rₙ(x,y) is the real part of the n′th complex high-resolution image at image point index x,y.

10. An ultrasound system configured to produce compounded ultrasound images by beamforming a first and a second low-resolution image using data from a first ultrasound emission, beamforming a third and a fourth low-resolution image using data from a second ultrasound emission, summing said first and said third low-resolution image creating a first high-resolution image and said second and said fourth low-resolution image creating a second high-resolution image, wherein the ultrasound system further is configured to compute a first envelope image for said first high-resolution image and a second envelope image for said second high-resolution image, and process said first envelope image and said second envelope image creating in a first compounded high-resolution image.
